(19) United States
(12) Patent Application Publication
(10) Pub. No.: US 2017/0105625 A1
(43) Pub. Date: Apr. 20, 2017

(54) Diagnostic Device of Optics Type for Breast

(71) Applicant: U BIOMED INC., Daegu (KR)

(72) Inventor: Nyeon Sik EUM, Daegu (KR)

(21) Appl. No.: 14/785,576

(22) PCT Filed: Sep. 4, 2014
(86) PCT No.: PCT/KR2014/008326

§ 371 Date: Oct. 19, 2015

(30) Foreign Application Priority Data
Jun. 24, 2014 (KR) 10-2014-0077290

(51) Int. Cl. A61B 5/00; A61B 10/00

(52) U.S. Cl. CPC A61B 5/0091; A61B 5/0075; A61B 5/0077; A61B 5/708; A61B 10/0041; A61B 2090/309

(57) ABSTRACT

The present invention relates to an optical breast diagnostic apparatus taking an image of a breast for diagnosing an abnormality in a female breast, including a hemispherical cover covering the breast with certain spacing; a light source installed inside the hemispherical cover; and a photographing device detachably installed on the hemispherical cover to take an infrared image of the breast. The optical breast diagnostic apparatus has an effect of objectively evaluating the diagnosis of breast cancer by obtaining an image from simple infrared imaging using a camera, without undergoing a complex process such as radiography.

### TECHNICAL FIELD

The present invention relates to an optical breast diagnostic apparatus, and more specifically to an optical breast diagnostic apparatus capable of objectively diagnosing breast cancer by installing a light source radiating infrared ray and a photographing device in a hemispherical cover covering a female breast and comparing an infrared image of breast taken by the photographing device with an image of normal breast.

### BACKGROUND ART

Worldwide, breast cancer is the leading type of cancer in women, accounting for 25% of all cases, and in Korea, the number of patients with breast cancer increased from 3,801 in 1996 to 16,398 in 2012, which is about a fourfold increase. According to statistics in 2013 from the Ministry of Health and Welfare, in national breast cancer screening over five years, among those tested positive, the number of patients diagnosed with cancer is 3,200, whereas among those tested negative, the number of patients diagnosed with cancer is 6,000. As such, the number of patients with cancer who were tested negative is twice as many as those tested positive. This suggests that patients tested negative also need to be continually monitored, and accordingly regular diagnosis is required.

For equipment for diagnosing breast cancer, mammographic X-ray apparatuses occupy 85.2% of the entire equipment market. However, in the case of regular screening, radiation exposure by X-rays may cause cancer, which is a disadvantage.

Also, a lump in a breast, which indicates the symptom of breast cancer, has various types. The most common type of malignant tumor is a hard and unmovable lump with no pain, and self breast exams may vary depending on patients' self exams skill and sensitivity felt from hands. Moreover, it is difficult to objectively record the degree of sensitivity felt from hands through self breast exams, and thus, a system allowing objective records is necessary.

### SUMMARY OF INVENTION

The present invention was invented to improve the above issues. It is an object of the present invention to provide an optical breast diagnostic apparatus including a light source radiating infrared ray with an infrared LED and a photographing device in a hemispherical cover covering a female breast, thereby allowing identification of a tissue such as tumor by distinguishing the tissue from a normal tissue through an infrared image obtained from taking an infrared photograph of the breast by the light source and photographing device.

In order to achieve the above object, an optical breast diagnostic apparatus taking an image of a breast for diagnosing an abnormality in a female breast of the present invention includes a hemispherical cover covering the breast with certain spacing; a light source installed inside the hemispherical cover; and a photographing device detachably installed on the hemispherical cover to take an infrared image of the breast.

Also, preferably, the edge of bottom of the hemispherical cover is formed with a contact part made of an elastic material, and the light source is an infrared LED or LD, or a lamp.

Also, preferably, the photographing device is any one of an infrared camera, a CCD camera, a moving picture camera, a digital camera, or a mobile phone camera.

Also, preferably, the photographing device is positioned at the centre of upper part of the hemispherical cover or in one part of side circumference of the hemispherical cover.

Also, preferably, the optical breast diagnostic apparatus further includes a partition installed between the light source inside the hemispherical cover and the photographing device, and the end of lower part of the partition is formed with a contact part made of an elastic material.

The optical breast diagnostic apparatus of the present invention has an effect of objectively evaluating the diagnosis of breast cancer which is tumor tissues such as those in the form of lump by obtaining an image from simple infrared imaging using a camera, without undergoing a complex process such as radiography.

Also, the optical breast diagnostic apparatus of the present invention is formed as a simple device and thus available for households, which enables individuals to frequently diagnose breast cancer readily without going to hospitals.

Also, the photographing device is detachable, and thus one of various cameras may be freely used depending on circumstances and a camera on a mobile phone such as a smart phone everyone has can be used for taking an image. Thus, the optical breast diagnostic apparatus of the present invention is convenient for use.

### BRIEF DESCRIPTION OF THE DRAWINGS

FIG. 1 is a cross-sectional view illustrating an optical breast diagnostic apparatus according to an embodiment of the present invention;

FIG. 2 is a cross-sectional view illustrating an optical breast diagnostic apparatus in use according to an embodiment of the present invention;

FIG. 3 is a cross-sectional view illustrating an optical breast diagnostic apparatus according to another embodiment of the present invention; and

FIG. 4 is a cross-sectional view illustrating an optical breast diagnostic apparatus in use according to another embodiment of the present invention.

### BEST MODE FOR CARRYING OUT THE INVENTION

Hereinafter, preferred embodiments of an optical breast diagnostic apparatus according to the present invention will be described in detail with reference to the accompanying drawings. The present invention is not limited to the following embodiments and may be implemented in different ways, and the embodiments are provided for complete disclosure and thorough understanding of the scope of the invention by persons having ordinary knowledge in the art.

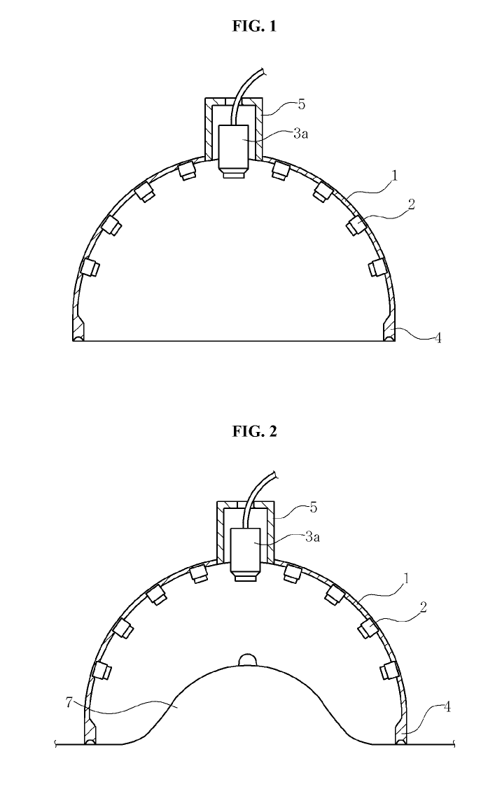

FIG. 1 is a cross-sectional view illustrating an optical breast diagnostic apparatus according to an embodiment of the present invention.

As illustrated in FIG. 1, the optical breast diagnostic apparatus according to an embodiment of the present invention includes a hemispherical cover 1, a light source 2, and a photographing device 3a, as basic elements.

The hemispherical cover 1 has a size suitable for fully covering a female breast, and since the cover needs to cover the breast with certain spacing, it is preferable to form the hemispherical cover slightly larger than the average size of breasts. Further, the edge of bottom of the hemispherical cover 1 is formed with a contact part 4 made of an elastic material such as rubber, so that the hemispherical cover 1 is brought into tight contact with the breast when covering the breast, and thereby, light from the light source 2 does not leak out.

The light source 2 is a type of a light in which multiple lights are attached inside the hemispherical cover 1 at regular intervals. Specifically, the light source is preferably an infrared LED or Laser Diode (LD), or a lamp, which radiates infrared ray, and serves as a provider of light reflected when taking an image of the breast with the photographing device 3a by this light source 2.

The photographing device 3a is a device taking an image of the breast and obtaining the image by receiving reflected light of incident light radiated from the light source 2. For the photographing device 3a, one of various types of cameras, such as an infrared camera, a CCD camera, a moving picture camera, a digital camera, or a mobile phone camera, is detachably installed at the centre of upper part of the hemispherical cover 1. In order to avoid leakage of light after installation of the photographing device 3a in the hemispherical cover 1, it is preferable to cover the rear part of the photographing device 3a with a separate cover 5.

### Modes for Carrying Out the Invention

FIG. 2 is a cross-sectional view illustrating an optical breast diagnostic apparatus in use according to an embodiment of the present invention.

As illustrated in FIG. 2, a breast 7 is fully covered with the hemispherical cover 1 to be brought into tight contact with a contact part 4, and infrared ray is radiated from the light source 2. Thereafter, the photographing device 3a takes an image of the breast 7, with reflected light obtained from incident light of radiated infrared ray in the breast 7, to obtain an infrared image. In the thus-obtained infrared image, a normal breast has the same illuminance throughout the image. However, when tumor tissues such as a lump which may be the symptom of breast cancer are present in the breast, these areas are dark in the infrared image of the breast, which is clearly distinguished in contrast from other areas of normal tissues. Thereby, the diagnosis of breast cancer can be readily made.

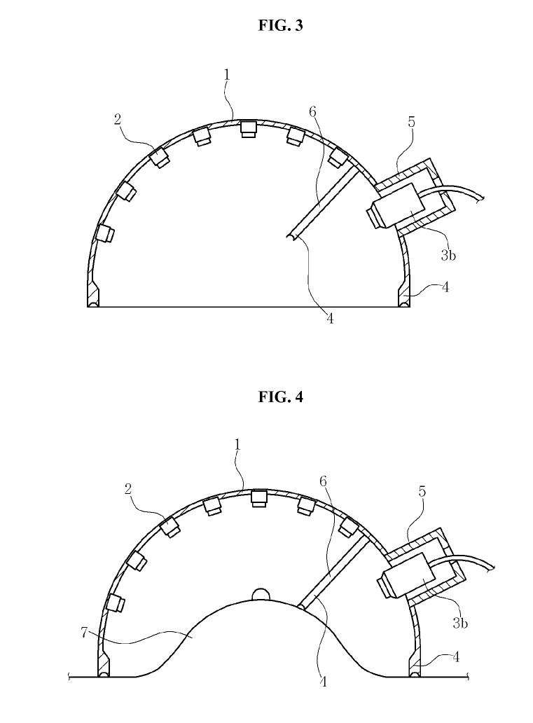

FIG. 3 is a cross-sectional view illustrating an optical breast diagnostic apparatus according to another embodiment of the present invention

As illustrated in FIG. 3, when the photographing device 3a is positioned at the center of upper part of the hemispherical cover 1, the infrared image may be blocked by a nipple and that area may not be clearly imaged. Since the photographing device is detachable, the location of the photographing device 3b may be moved to one part of side circumference of the hemispherical cover 1. Here, the photographing device is removed from the center of upper part of the hemispherical cover, and thus the center of upper part of the hemispherical cover becomes open. Therefore, it is preferable to separately cover the open area.

When the photographing device 3b is installed on side circumference of the hemispherical cover 1, a partition 6 is installed between the photographing device 3b and the light source 2, so as not to be affected by incident light of infrared ray radiated from the light source 2. Further, it is preferable to form the end of lower part of the partition 6 with a contact part 4 made of an elastic material such as rubber, so as to avoid leakage of light, and it is preferable to cover the rear part of the photographing device 3b with a separate cover 5.

FIG. 4 is a cross-sectional view illustrating an optical breast diagnostic apparatus in use according to another embodiment of the present invention.

As illustrated in FIG. 4, when covering the breast 7 with the hemispherical cover 1, the photographing device 3b is installed on the side circumference of the hemispherical cover, and the partition 6 prevents incident light directly radiated from the light source 2 from entering the photographing device 3b. When taking an image of the breast by the photographing device 3b in a state where the hemispherical cover 1 is rotated, for example, every 90°, according to the optical breast diagnostic apparatus of another embodiment, infrared images taken by receiving reflected light of infrared ray radiated from the light source 2 can be obtained from various positions around the breast 7. This allows more accurate diagnosis of breast cancer.

Thus, the thus-obtained infrared images enable identification of tumor tissues such as lump using a method as above and simple diagnosis of breast cancer.

The optical breast diagnostic apparatus according to the present invention is described with referenced to the drawings. However, the present invention is not limited to the disclosed embodiments and drawings, and various modifications may be made by a person skilled in the art within the scope of the spirit of the present invention.

The present invention can be useful for objectively evaluating diagnosis of breast cancer which is tumor tissues such as those in the form of lump by obtaining an image from simple infrared imaging using a camera, without undergoing a complex process such as radiography.

Also, the present invention is formed as a simple device and thus available for households, which enables individuals to effectively use the present invention for frequently diagnosing breast cancer readily, without having to go to hospitals.

Also, since the photographing device is detachable, one of various cameras may be freely used depending on circumstances, and moreover a camera on a mobile phone such as a smart phone everyone has can be used for taking an image. Thus, the present invention can be useful.

1. An optical breast diagnostic apparatus for taking an image of a breast to diagnose an abnormality in a female breast, comprising:
a hemispherical cover to cover the breast with certain spacing;
a light source installed inside the hemispherical cover; and
a photographing device to take an infrared image of the breast, the device detachably installed on the hemispherical cover.

2. The optical breast diagnostic apparatus of claim 1, wherein the edge of bottom of the hemispherical cover is formed with a contact part made of an elastic material.

3. The optical breast diagnostic apparatus of claim 1, wherein the light source is an infrared LED or LD, or a lamp.

4. The optical breast diagnostic apparatus of claim 1, wherein the photographing device is any one of an infrared camera, a CCD camera, a moving picture camera, a digital camera, or a mobile phone camera.

5. The optical breast diagnostic apparatus of claim 1, wherein the photographing device is positioned at the center of upper part of the hemispherical cover.

6. The optical breast diagnostic apparatus of claim 1, wherein the photographing device is positioned in one part of side circumference of the hemispherical cover.

7. The optical breast diagnostic apparatus of claim 6, further comprising a partition installed between the light source inside the hemispherical cover and the photographing device.

8. The optical breast diagnostic apparatus of claim 7, wherein the end of lower part of the partition is formed with a contact part made of an elastic material.
